(19) United States
(12) Patent Application Publication
(10) Pub. No.: US 2026/0102323 A1
(43) Pub. Date: Apr. 16, 2026

(54) STABLE COSMETIC COMPOSITION WITH HIGH LEVEL OF ELECTROLYTES

(71) Applicant: L'OREAL, Paris (FR)

(72) Inventors: Rebecca CHEN, Princeton, NJ (US); Brian CHING, Clark, NJ (US)

(73) Assignee: L'OREAL, Paris (FR)

(21) Appl. No.: 18/916,321

(22) Filed: Oct. 15, 2024

(51) Int. Cl. A61K 8/44; A61K 8/25; A61Q 19/00

(52) U.S. Cl. CPC A61K 8/44; A61K 8/26; A61K 8/73; A61K 8/8182; A61Q 19/00; A61K 2800/48; A61K 2800/52; A61K 2800/5424; A61K 2800/591

(57) ABSTRACT

The instant disclosure is drawn to a cosmetic or personal care composition comprising: a) at least one amino acid; b) at least one clay compound; c) at least one anionic thickening polymer; and wherein the composition is thermally stable and contains a high level of electrolytes.

### FIELD OF THE DISCLOSURE

The present disclosure relates to cosmetic and personal care compositions comprising high level of electrolytes.

### BACKGROUND

A common problem associated with formulating compositions, especially composition comprising active ingredients, is ensuring physical stability. Many active ingredients, including electrolytes, can cause stability problems, especially when used in high amounts. The consequence of stability problems is significant. For example, stability problems can cause partial, if not complete, loss of product integrity, color loss, malodor, viscosity changes, etc. With respect to active ingredients, stability problems reduce or eliminate activity, and prevent the active ingredients from reaching their intended target.

There is a need for compositions that are physically stable and effective. The inventors of the instant case found that a unique combination of certain polymers, clays and amino acids resulted in a rheological-modifying and stabilizing polymer blend.

### SUMMARY OF THE DISCLOSURE

The instant disclosure relates to cosmetic compositions comprising the association of amino acids, clays and anionic thickening polymers. The cosmetic compositions provide thermally stable compositions even though they contain high level of electrolytes.

The composition includes a unique combination of amino acids, clays and anionic thickening polymers that provide thermally stable compositions even though there is a high level of electrolytes.

The composition typically includes:
a) at least one amino acid;
b) at least one clay compound;
c) at least one anionic thickening polymer; and
wherein the composition is thermally stable and contains a high level of electrolytes.

In some embodiments, the at least one amino acid has an isoelectric point value above 7. Nonlimiting examples of one amino acid include arginine, lysine and mixtures thereof. In some embodiments, the at least one amino acid is present from about 0.5 to about 4% wt. based on the total weight of the composition.

Nonlimiting examples of clay compounds include hectorite, smectite, kaolin, bentonite, montmorillonite, or mixture thereof. In some embodiments, the at least clay compound is present from about 0.1 to about 0.5% wt. based on the total weight of the composition.

In some embodiments, the at least one anionic thickening polymer is chosen from polymers and/or copolymers of 2-acrylamido-2-methylpropanesulfonic acid, polysaccharide polymers and mixtures thereof. In various embodiments, the at least one anionic thickening polymer is chosen from ammonium acryloyldimethyltaurate/VP Copolymer, xanthan gum, and mixture thereof. In some embodiments, the at least one anionic thickening polymer is present from about 0.3 to about 2% wt. based on the total weight of the composition.

In various embodiments, the at least one electrolyte is chosen from the group of α- and β-hydroxy acids, carboxylic acids, taurine, phosphoric acids, pyrophosphoric acid, cosmetically active organic acids, and mixture thereof. In some embodiments, the at least one electrolyte is chosen from glycolic acid, lactic acid, malic acid, citric acid, tartaric acid, mandelic acid, gluconic acid, ascorbic acid, and mixtures thereof. In some embodiments, the at least one electrolyte is present from about X % wt. to about Y % wt. based on the total weight of the composition.

In some embodiments, the compositions may have a pH of about 5 to about 6.

In a different aspect, the composition may include:
a) At least one amino acid;
b) At least one clay compound;
c) At least one anionic thickening polymer chosen from polymers and/or copolymers of 2-acrylamido-2-methylpropanesulfonic acid, anionic polysaccharide polymers and mixtures thereof; and wherein the composition is thermally stable and contains a high level of electrolytes.

In another aspect, the composition may include:
a) arginine;
b) at least one clay compound;
d) at least one anionic thickening polymer; and
wherein the composition is thermally stable and contains a high level of electrolytes.

The instant disclosure is also directed to methods for treating the keratin materials, preferably the skin, more preferably the face, comprising applying the cosmetic composition to said keratin materials.

Without being bound by theory or mechanism, it is suggested that the unique combination of amino acids, clays and anionic thickening polymers provide a synergistic combination that thermally stabilized compositions even though there is presence of a high level of electrolytes.

It is to be understood that both the foregoing general description and the following detailed description are exemplary and explanatory only, and are not restrictive of the invention.

### DETAILED DESCRIPTION OF THE DISCLOSURE

The composition includes a unique combination of amino acids, clays and anionic thickening polymers that provide a synergistic combination that thermally stabilized the compositions even though there is presence of a high level of electrolytes.

The composition typically includes:
a) at least one amino acid;
b) at least one clay compound;
c) at least one anionic thickening polymer; and
wherein the composition is thermally stable and contains a high level of electrolytes.

### Amino Acids

In accordance with the various embodiments, the composition according to the disclosure comprises amino acids.

In some embodiments, the at least one amino acid has an isoelectric point value above 7. Nonlimiting examples of amino acid include arginine, lysine and mixtures thereof. In accordance with the various embodiments, the composition according to the disclosure comprises arginine.

In accordance with the various embodiments, the amount of the at least one amino acid in the composition may be from about 0.5 to about 4 wt. %, or from about 0.45 to about 3.95 wt. %, or from about 0.4 to about 3.90 wt. %, or from about 0.3 to about 3.8 wt. %, or from about 0.4 to about 3.7 wt. %, or from about 0.5 to about 3.6 wt. %, or from about 0.6 to about 3.5 wt. %, or from about 0.7 wt. % to about 3.4 wt. %, or from about 0.8 to about 3.3 wt. %, or from about 0.9 to about 3.2 wt. % or any suitable combination, sub-combination, range, or sub-range thereof by weight, based on the total weight of the composition.

Thus, any one of or a combination of amino acid is present, by weight, based on the total weight of the composition, from about 0.5, 0.6, 0.7, 0.8, 0.9, 1.0, 1.1, 1.2, 1.3, 1.4, 1.5, 1.6, 1.7, 1.8, 1.9, 2.0, to about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9 or 4 wt. % including increments and ranges therein and there between.

### Clays Compounds

In various embodiments, the at least one clay compound may be chosen from kaolin, bentonite, smectite, hectorite, vermiculite, illite, chorite, halloysite, palygorskite, sepiolite, sesquioxide, imogolite, allophane, or combinations of two or more thereof. For example, in some embodiments, the clay compound comprises, consists essentially of, or consists of smectite, hectorite, kaolin, bentonite, or a combination of one or more thereof. The solvent may be chosen from water and/or non-aqueous solvents.

In various embodiments, the total amount of clay compounds can range from about 0.1% to about 0.5% wt. %, such as from about 0.15% to about 0.5% wt. %, from about 0.15% to about 0.45% wt. % or any suitable combination, sub-combination, range, or sub-range thereof by weight, based on the total weight of the composition.

Thus, any one of or a combination of clay compound is present, by weight, based on the total weight of the composition, from about 0.1, 0.15, 0.2, 0.25, 0.3, 0.35, 0.4, 0.45, or 0.5 wt. % including increments and ranges therein and there between.

Use can in particular be made, as unmodified clay and more particularly as unmodified hectorite, of that sold by Elementis under the name Bentone Hydroclay 2000 LO.

### Anionic Thickening Polymer

The composition according to the invention comprises at least one anionic thickening polymer chosen from polyacrylamide polymers, such as 2-acrylamido-2-methylpropanesulfonic acid homopolymers and/or copolymers, gums or polysaccharide polymers and mixtures thereof. In some embodiments, the at least one anionic thickening polymer is chosen from polymers and/or copolymers of 2-acrylamido-2-methylpropanesulfonic acid, polysaccharide polymers and mixtures thereof. In various embodiments, the at least one anionic thickening polymer is chosen from ammonium acryloyldimethyltaurate/VP Copolymer, xanthan gum, and mixture thereof.

The polyacrylamide polymers that may be suitable for use in the context of the present invention may be anionic polyacrylamide polymers, including branched or unbranched substituted polymers.

Among the polyacrylamides, examples that may be mentioned include the crosslinked copolymers sold under the names Sepigel 305 (CTFA name: Polyacrylamide/C13-14 isoparaffin/Laureth 7) or Simulgel 600 (CTFA name: Acrylamide/sodium acryloyldimethyltaurate copolymer/isohexadecane/polysorbate 80) by the company SEPPIC.

Among the 2-acrylamido-2-methylpropanesulfonic acid copolymers, mention may be made of ammonium polyacryloyldimethyltaurate, such as the product sold under the name Hostacerin AMPS by the company Clariant, and ammonium acryloyldimethyltaurate/VP copolymer, such as the product sold under the name Aristoflex AVC by the company Clariant.

Other polyacrylamide polymers that may be used in the context of the present invention include multiblock copolymers of acrylamides and of acrylamides substituted with acrylic acids and with substituted acrylic acids. Among the commercially available examples of these multiblock copolymers, mention may be made of acryloyldimethyltaurate/steareth-25 methacrylate crosspolymer such as the product sold by the company Clariant under the name Aristoflex HMS and the copolymers sold under the names Hypan SR150H, SS500V, SS500W, SSA100H, from Lipo Chemicals, Inc.

According to a particular embodiment of the invention, the polyacrylamide polymers or copolymers that are suitable for use in the invention are chosen from ammonium acryloyldimethyltaurate/VP copolymer and acryloyldimethyltaurate/steareth-25 methacrylate copolymer.

A wide variety of gums and polysaccharides may be used in the context of the present invention as thickening polymers.

“Polysaccharides” are gelling agents which contain a backbone of repeating sugar (i.e. carbohydrate) units. Non-limiting examples of polysaccharide thickening polymers include those chosen from the group consisting of cellulose, carboxymethylhydroxyethylcellulose, cellulose acetate propionate carboxylate, hydroxyethylcellulose, hydroxyethylethylcellulose, hydroxypropylcellulose, hydroxypropylmethylcellulose, methylhydroxyethylcellulose, microcrystalline cellulose, sodium cellulose sulfate and mixtures thereof.

Alkyl-substituted celluloses are also suitable. Among the alkylhydroxyalkylcellulose ethers, mention may be made of the starting material denoted under the name cetylhydroxyethylcellulose, namely a mixture of cetyl alcohol ether and hydroxyethylcellulose. This starting material is notably sold under the brand name Natrosol® CS Plus from Aqualon Corporation. Other polysaccharides include starch derivatives (for example, starch oxide, starch dialdehyde, dextrin, British gum, acetyl starch, starch phosphate, carboxymethyl starch, hydroxyethyl starch, hydroxypropyl starch).

Celluloses that may notably be mentioned include quaternized alkylhydroxyethylcelluloses containing C8-C30 fatty chains, such as the products Quatrisoft LM 200, Quatrisoft LM-X 529-18-A, Quatrisoft LM-X 529-18B (C12 alkyl) and Quatrisoft LM-X 529-8 (C18 alkyl) sold by the company Amerchol and the products Crodacel QM, Crodacel QL (C12 alkyl) and Crodacel QS (C18 alkyl) sold by the company Croda.

Among the cellulose derivatives, mention may also be made of celluloses modified with groups including at least one fatty chain, such as hydroxyethylcelluloses modified with groups including at least one fatty chain such as alkyl groups, notably of C8-C22, arylalkyl, alkylaryl groups, such as Natrosol Plus Grade 330 CS (C16 alkyl) sold by the company Aqualon, and celluloses modified with polyalkylene glycol alkyl phenyl ether groups, such as the product Amercell Polymer HM-1500 (polyethylene glycol (15) nonyl phenyl ether) sold by the company Amerchol.

The celluloses and derivatives may also be sold under the names Avicel® (microcrystalline cellulose, MCC) by the company FMC Biopolymers, under the name Cekol (carboxymethylcellulose) by the company Noviant (CP-Kelco), under the name Akucell AF (sodium carboxymethylcellulose) by the company AkzoNobel, under the name Methocel™ (cellulose ethers) and Ethocel™ (ethylcellulose) by the company Dow, and under the names Aqualon® (carboxymethylcellulose and sodium carboxymethylcellulose), Benecel® (methylcellulose), Blanose™ (carboxymethylcellulose), Culminal® (methylcellulose, hydroxypropylmethylcellulose), Klucel® (hydroxypropylcellulose), Polysurf® (cetylhydroxyethylcellulose) and Natrosol® CS (hydroxyethylcellulose) by the company Hercules Aqualon.

The polysaccharide polymers that are suitable for use in the invention may be chosen from xanthan gums. Xanthan gums are sold, for example, under the name Rhodicare by the company Rhodia Chimie, under the name Satiaxane™ by the company Cargill Texturizing Solutions (for the food, cosmetic and pharmaceutical industries), under the name Novaxan™ by the company ADM, and under the names Kelzan® and Keltrol® (for example Keltrol® CG) by the company CP-Kelco.

According to a particular embodiment of the disclosure, the polysaccharide polymers that are suitable for use in the disclosure are chosen from cellulose, xanthan gum, and mixtures thereof.

In various embodiments, the total amount of anionic thickening polymer can range from about 0.1% to about 0.5% wt. %, such as from about 0.15% to about 0.5% wt. %, from about 0.15% to about 0.45% wt. % or any suitable combination, sub-combination, range, or sub-range thereof by weight, based on the total weight of the composition.

Thus, any one of or a combination of anionic thickening polymer is present, by weight, based on the total weight of the composition, from about 0.1, 0.15, 0.2, 0.25, 0.3, 0.35, 0.4, 0.45, or 0.5 wt. % including increments and ranges therein and there between.

### Electrolytes

The electrolyte may be a cosmetically active ingredient for keratinous substances, in particular skin.

The electrolyte may be selected from organic acids and salts thereof.

The electrolyte may be selected from hydroxy acids, such as α- and β-hydroxy acids, carboxylic acids, taurine, phosphoric acids, pyrophosphoric acid, and cosmetically active organic acids, such as ascorbic acid, glycyrrhizic acid, and UV absorbers, for example, 2-hydroxy-4-methoxybenzophenone-5-sulfonic acid, terephthalylidene dicamphor sulfonic acid and phenylbenzimidazole sulfonic acid.

The electrolyte may be selected from ascorbic acid and salts thereof.

The composition according to the present invention comprises (a) at least one electrolyte. Two or more electrolytes may be used in combination. Thus, a single type of electrolyte or a combination of different types of electrolytes may be used.

The electrolyte may be a cosmetically active ingredient for keratinous substances, in particular skin, such as anti-aging agents, whitening agents, anti-wrinkle agents, moisturizing agents, antiphlogistic agents, astringent agents, ultraviolet absorbers, and/or antiperspirant agents. In the present invention, the (a) electrolytes are not particularly limited as long as they can be used as raw materials for cosmetics, and may be appropriately blended depending on the intended purpose.

The electrolyte may include, for example, organic acids and salts thereof, and inorganic acids and salts thereof. Preferably, the electrolyte may be selected from organic acids and salts thereof.

The term “organic acid” here means nonpolymeric organic compounds showing acidic property when it dissolves in water. The solubility in water of the organic acid is not particularly limited, but in general, at least 1 g/100 mL water, preferably at least 5 g/100 mL water, and in particular at least 10 g/100 ml water at room temperature (25° C.) under atmospheric pressure (760 mmHg).

The organic acid may have a mean molecular weight by weight less than 500, preferably less than 400, in particular less than 300, and more particularly less than 250. In the context of the present specification, the mean molecular weight indicates a number average molecular weight.

The organic acid may be selected from, for example, hydroxy acids, such as α- and B-hydroxy acids, carboxylic acids, amino acids, taurine, phosphoric acids, pyrophosphoric acid, and cosmetically active organic acids, such as ascorbic acid, glycyrrhizic acid, and UV absorbers, for example, 2-hydroxy-4-methoxybenzophenone-5-sulfonic acid, terephthalylidene dicamphor sulfonic acid and phenylbenzimidazole sulfonic acid.

Regarding α- and β-hydroxy acids, the α and β positions reflect the fact that at least one of the hydroxyl functions occupies an α or β position relative to at least one of the carboxyl functions of the acid, i.e., is attached, respectively, either to the carbon bearing the hydroxyl function or to the carbon adjacent to the one bearing the carboxyl function. The term “a-hydroxy acid”, or “AHA”, here means a carboxylic acid which has at least one hydroxyl group on the adjacent (alpha) carbon atom. The term “β-hydroxy acid”, or “BHA”, here means a carboxylic acid which has at least one hydroxyl group on the beta carbon atom.

The α-hydroxy acid may be represented by the following chemical formula:

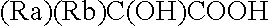

where
Ra and Rb are H, F, Cl, Br, I, alkyl, aralkyl or aryl group of saturated or unsaturated, isomeric or non-isomeric, straight or branched chain or cyclic form, having 1 to 25 carbon atoms, and in addition Ra and Rb may carry OH, CHO, COOH and alkoxyl group having 1 to 9 carbon atoms.

The hydrogen atom attached to the carbon atom may be substituted by F, Cl, Br, I, or lower alkyl, aralkyl, aryl or alkoxyl group having 1 to 9 carbon atoms. The alpha hydroxyacids may be present as a free acid or lactone form, or in a partial salt form with an organic base or an inorganic alkali. The alpha hydroxyacids may exist as stereoisomers such as D, L, DL and meso forms.

Typical alkyl, aralkyl, aryl and alkoxyl groups for Ra and Rb include methyl, ethyl, propyl, propyl, isopropyl, butyl, pentyl, benzyl, phenyl, methoxyl, and ethoxyl.

The α-hydroxy acid may be selected from, for example, the group consisting of glycolic acid, lactic acid, malic acid, citric acid, tartaric acid, mandelic acid, gluconic acid and mixtures thereof, preferably from the group consisting of glycolic acid, lactic acid, citric acid, tartaric acid, and mixtures thereof, more preferably from lactic acid, citric acid, tartaric acid, and mixtures thereof.

As β-hydroxy acids, mention may be made, without limitation, of salicylic acid and its derivatives.

The carboxylic acid may be selected from C1-C6 monocarboxylic acids, such as acetic acid, propanoic acid, butanoic acid; C1-C6 dicarboxylic acids; such as maleic acid, succinic acid, glutaric acid, adipic acid, oxalic acid, and malonic acid; aromatic carboxylic acids, such as phthalic acid and salicylic acid; pyruvic acid, and glucuronic acid.

The cation moiety of the electrolyte may be metallic cations or organic cations. As metallic cations, mention can be made of alkali metal ions and alkaline earth metal ions, such as Na+, K+, Ca2+, and Mg2+, as well as Zn2+ and Al3+. As organic cations, mention can be made of ammonium ion, sulfonium ion, phosphonium ion, tertiary amine such as trimethylamine salts, triethylamine salts, monoethanolamine salts, triethanolamine salts and pyridine salts.

As the electrolyte, mention can be made of the following organic acids and inorganic salts thereof, such as calcium salts, potassium salts, sodium salts, and magnesium salts: lactic acid and lactates, such as sodium lactate, magnesium lactate and calcium lactate; phosphoric acid and phosphate, such as potassium phosphate, sodium phosphate, sodium pyrophosphate; glycyrrhizin and glycyrrhizinate, such as dipotassium glycyrrhizinate; ascorbic acid and salts thereof, such as magnesium ascorbate phosphate, sodium ascorbate phosphate, sodium ascorbate, disodium L-ascorbyl sulfate; pyrrolidone carboxylic acid and pyrrolidone carboxylates, such as sodium pyrrolidone carboxylate and zinc pyrrolidone carboxylate; amino acids such as proline, aspartic acid, glutamic acid, serine and their derivatives; UV absorbers such as 2-hydroxy-4-methoxybenzophenone-5-sulfonic acid and sodium salts thereof, and the like. In particular, the (a) electrolyte is selected from ascorbic acid and salts thereof.

The amount of the electrolyte(s) in the composition according to the instant disclosure is at least 3 wt. % based on the total weight of the composition.

The electrolyte(s) may be present in a content of 7% by weight or more, preferably 8.5% by weight or more, and more preferably 10% by weight or more, relative to the total weight of the composition.

The electrolyte(s) may be present in a content of 30% by weight or less, preferably 20% by weight or less, and more preferably 10% by weight or less, relative to the total weight of the composition.

The electrolyte(s) may be present in a content ranging from 3% to 30% by weight, preferably from 4% to 29% by weight, preferably from 5% to 28% by weight, preferably, from 6% to 28% by weight, preferably from 7% to 25% by weight, preferably from 8.5% to 20% by weight, and even more preferably from 9% to 15% by weight, relative to the total weight of the composition.

pH

The pH of the cosmetic composition may vary. In some instances, the pH of the cosmetic composition is from about 5 to about 6. Moreover, in some cases, the pH of the cosmetic composition is from 5 to 5.5. Furthermore, in some instances, the pH of the cosmetic composition is below 6, for example, 5.8 or less, 5.5 or less, 5.2 or less.

### Optional Actives and Other Ingredients

In some embodiments, there may be one or more optional actives or other ingredients (herein, “additives”) present in the salt-stable cosmetic composition, the one or more additives selected from, for example, but not limited to: humectants, such as acetamide MEA, glycols, such as glycerin and propylene glycol; anti-microbials; antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria baicalensis root extract), pine bark extract (Pinus pinaster bark/bud extract), ellagic acid; hyaluronic acid and its derivatives; hydroxyacetophenone; and vitamins and vitamin derivatives, such as tocopherol and ascorbic acid; and combinations thereof.

In some embodiments, additives may include one or a combination of antimicrobial agents and their salts, for example, including, but not limited to, the group consisting of chlorphenesin, caprylyl glycol, phenoxyethanol, caprylhydroxamic acid, benzoic acid, salicylic acid, benzyl alcohol, phenethyl alcohol, benzalkonium chloride, 4-hydroxyacetophenone, piroctone olamine, hexyl glycerin, ethylhexylglycerin, octylglycerin, benzylglycerin, 3-heptoyl-2,2-propandiol, and 1,2-hexandiol, and pentylene glycol, and combinations thereof.

In some embodiments, additives may include one or a combination of skin actives, for example, including, but not limited to, the group consisting of tocopherol, phenoxyethanol, sodium hyaluronate, capryloyl salicylic acid, phenylethyl resorcinol, hydroxyacetophenone, and combinations thereof.

In some embodiments, the one or more additives present in the salt-stable cosmetic composition may include one or more other components, for example, including, but not limited to, the group consisting of penetrants; sequestrants; fragrances; dispersants; ceramides; opacifiers and combinations thereof. Although the aforementioned optional components are given as an example, it will be appreciated that other optional components compatible with cosmetic applications known in the art may be used.

In accordance with the various embodiments, the amounts of additives, for example, actives and other components, present in the salt-stable cosmetic composition can range from about 0.001% to about 50%, from about 0.5% to about 30%, from about 1.5% to about 20%, and from about 5% to about 15%, or any suitable combination, sub-combination, range, or sub-range thereof by weight, based on the weight of the oxidizing component or the salt-stable cosmetic composition.

In some embodiments, one or more additives, alone or in combination, can be present in one or both of the oxidizing component and the salt-stable cosmetic composition from about 0.05% to about 50% by weight, from about 0.05% to about 2.5% by weight, from about 0.1% to about 2%, from about 0.25% to about 1.5%, and from about 0.5% to about 1.25%, or any suitable combination, sub-combination, range, or sub-range thereof by weight, based on the weight of the salt-stable cosmetic composition.

In some embodiments, one or more other components, such as preservatives, vitamins, preservatives, and the like, alone or in combination, can be present in one or both of the oxidizing component and the salt-stable cosmetic composition from about 0.05% to about 50% by weight, from about 0.05% to about 25% by weight, from about 0.1% to about 10%, from about 0.25% to about 5%, and from about 0.5 to about 3.5%, or any suitable combination, sub-combination, range, or sub-range thereof by weight, based on the weight of the salt-stable cosmetic composition.

Thus, one or a combination of additives may be present in the salt-stable cosmetic composition, by weight, based on the weight of the oxidizing component or the salt-stable cosmetic composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

### Cosmetically Acceptable Carrier System

The cosmetic compositions include a cosmetically acceptable carrier system. The term “cosmetically acceptable” means a carrier that is compatible with any keratinous substrate, and for purposes hereof, includes water and optionally water based solvents subject to any exclusions as disclosed herein.

The cosmetic compositions may comprise any constituent normally employed in the topical application and administration envisaged. Mention may in particular be made of water, solvents, fatty compounds (i.e. described by the International Federation Societies of Cosmetic Chemists, for example, in Cosmetic Raw Material Analysis and Quality, Volume I: Hydrocarbons, Glycerides, Waxes and Other Esters (Redwood Books, 1994), which is incorporated herein by reference in its entirety), polyols, pigments, fillers, silicones, surfactants, thickeners, gelling agents, preservatives and their mixtures in all proportions.

### Methods of Use

The instant disclosure also relates to methods of using the cosmetic compositions described herein. For example, the cosmetic compositions can be used in a method that comprises applying the cosmetic compositions to the skin of humans. In some cases, the composition is applied to the face. Furthermore, the cosmetic composition can be used in methods for depigmenting and/or bleaching keratin materials, preferably skin, comprising the step of: applying to the keratin substance the composition according to the compositions described herein. The aforementioned methods are non-therapeutic.

The instant disclosure also relates to non-therapeutic cosmetic process for depigmenting, lightening and/or bleaching keratin materials, preferably skin, comprising the step of applying to the keratin substance the composition according to the compositions described herein.

### EXAMPLES

Implementation of the present disclosure is provided by way of the following examples. The following examples serve to elucidate aspects of the technology without being limiting in nature.

### Example

The objective of the instant disclosure is to use smectite clays and amino acids to address the thermal stability challenge associated with thickening system in composition with high level of electrolytes.

### Demonstration of the Synergy Between Smectite Clays and Amino Acids in Improving Thermal Stability (in Solution)

TABLE 1
Example 1

| Example 1 | INCI Name | % (active) |
|---|---|---|
| Smectite clay | Hectorite | 0.24 |
| Amino Acid | Arginine | 1.80 |
| Polymer | Xanthan Gum | 0.36 |
| Electrolyte | Ascorbic | 8.4 |
| solution | Acid | |
| Other actives | | 4 |
| PH adjuster | Hydroxide | |
| | (pH 6) | |
| | Glycerin | 12 |

### Table 2: Results

To demonstrate the effect of the association between smectite clays and amino acid when they are in presence of a high amount of electrolyte, we assessed the percentage change in viscosity at a low shear rate region (0.1 1/s) after storing the mixture at 60° C. for one week. The rheological behavior at low shear rates was indicative of the storage stability of the formulation. In Table 2, we have shown that before the thermal treatment, the composition with xanthan gum alone was comparable with the one with additional arginine and hectorite. However, after 1-week thermal treatment at 60° C., the viscosity of composition with xanthan gum alone reduced by close to 25%, while the one with addition of arginine and hectorite only experienced around 6.5% viscosity decrease. Although xanthan gum with addition of hectorite also improved the thermal stability slightly, the synergy among the three is very clear.

| | | 1 week 60° | % |
|---|---|---|---|
| | T0 @0.1 1/s | C. @ 0.1 1/s | change |
| Xanthan Gum | 17.4355 (Pa · s) | 13.1496 | −24.58 |
| Xanthan Gum + | 14.4874 | 10.7828 | −25.57 |
| Arginine | | | |
| Xanthan Gum + | 18.8728 | 15.0784 | −20.11 |
| Hectorite | | | |
| Xanthan Gum + | 17.5304 | 16.3984 | −6.46 |
| Arginine + Hectorite | | | |

TABLE 3
Example 2

| | INCI Name | % |
|---|---|---|
| Smectite clay | Hectorite | 0.24 |
| Amino Acid | Arginine | 1.8 |
| Polymer | Aristoflex AVC | 1.8 |
| Electrolyte | Ascorbic | 8.4 |
| solution | Acid | |
| Other actives | | 4 |

### Table 4: Results

To demonstrate the effect of the association between smectite clays and amino acid when they are in presence of a high amount of electrolyte, we assessed the percentage change in viscosity at a low shear rate region (0.1 1/s) after storing the mixture at 60° C. for one week. The rheological behavior at low shear rates is indicative of the storage stability of the formulation. The rheological behaviors for compositions with Aristoflex AVC alone, with Arginine, with Hectorite and with Argine and Hectorite were recorded in Table 4.

In Table 4, after 1-week thermal treatment at 60° C., the viscosity of composition with Aristoflex AVC alone was reduced by close to 11%. When the compositions contained Arginine or Hectorite, the change in viscosity was less than with Aristoflex alone and was about 4.7% with Arginine and 3.7% with Hectorite. However, it was shown that the composition that contained both, Arginine and Hectorite, had a dramatic change in viscosity. Although Aristoflex AVC with addition of hectorite improved the thermal stability slightly, the synergy that appear when Aristoflex AVC was combined with Arginine and Hectorite was very clear.

TABLE 4

| | | 1 week 60° | % |
|---|---|---|---|
| | T0 @0.1 1/s | C. @ 0.1 1/s | change |
| Aristoflex AVC | 37.98 (Pa · s) | 33.83 | −10.93 |
| Aristoflex AVC + | 33.7 | 35.28 | −4.69 |
| Arginine | | | |
| Aristoflex AVC + | 79.02 | 76.1 | −3.7 |
| Hectorite | | | |
| Aristoflex AVC + | 125.15 | 162.21 | 29.61 |
| Arginine + Hectorite | | | |

### Demonstration of Synergy Between Hectorite and Arginine in Improving Thermal Stability (in Gel)

TABLE 5
Example 3

| | INCI Name | % |
|---|---|---|
| Smectite clay | Hectorite | 0.24 |
| Amino Acid | Arginine | 1.8 |
| Polymer | XanthanGum | 0.72 |
| Electolyte | Ascorbic | 8.4 |
| solution | Acid | |
| Other actives | | 4 |

### Table 6: Results

To demonstrate the effect of the association between smectite clays and amino acid when they are in presence of a high amount of electrolyte, we assessed the percentage change in viscosity at a low shear rate region (0.1 1/s) after storing the mixture at 60° C. for one week. The rheological behavior at low shear rates was indicative of the storage stability of the formulation. In Table 6, we've shown that after thermal treatment at 60° C. for 1 week, the solution with Xanthan Gum on its own shows a 4.15% decrease in viscosity. The combination of Xanthan Gum and Hectorite was slightly worse, showing a 7.04% decrease. Though the combination of Xanthan Gum and Arginine showed very slight improvement over just Xanthan Gum alone, the combination of Xanthan Gum, Arginine and Hectorite yielded the best result, managing to resist any decrease in viscosity and even showing a 5.71% increase after thermal treatment.

| | | 1 week 60° | % |
|---|---|---|---|
| | T0 @0.1 1/s | C. @ 0.1 1/s | change |
| XanthanGum | 59.28 (Pa · s) | 56.82 | −4.15 |
| Xanthan Gum + | 60.07 | 60.49 | 0.7 |
| Arginine | | | |
| Xanthan Gum + | 63.95 | 59.45 | −7.04 |
| Hectorite | | | |
| Xanthan Gum + | 60.37 | 63.82 | 5.71 |
| Arginine + Hectorite | | | |

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions can be modified in all instances by the term “about,” meaning within +/−10% of the indicated number. For example, an amount of “about 1 wt.” can include an amount as low as 0.90 wt. % or as high as 1.1. Similarly, an amount of “about 50” can include an amount as low as 45 wt. % and as high as 55 wt. %.

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both an emollient and a nonionic surfactant. If a particular composition includes both an emollient and a nonionic surfactant, a single compound will serve as only the emollient or only as the nonionic surfactant (the single compound does not simultaneously serve as both the emollient and nonionic surfactant).

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A composition comprising:
a) at least one amino acid;
b) at least one clay compound;
c) at least one anionic thickening polymer; and
wherein the composition is thermally stable and contains a high level of electrolytes.

2. The composition of claim 1, wherein the at least one amino acid is chosen from amino acids with an isoelectric point value above 7.

3. The composition of claim 1, wherein the at least one amino acid is chosen from arginine, lysine and mixtures thereof.

4. The composition of claim 1, wherein the at least one amino acid is present from about 0.5 to about 4% wt. based on the total weight of the composition.

5. The composition of claim 1, wherein the at least one clay compound is chosen from hectorite, smectite, kaolin, bentonite, montmorillonite, or mixture thereof.

6. The composition of claim 1, wherein the at least clay compound is present from about 0.1 to about 0.5% wt. based on the total weight of the composition.

7. The composition of claim 1, wherein the at least one anionic thickening polymer is chosen from polymers and/or copolymers of 2-acrylamido-2-methylpropanesulfonic acid, polysaccharide polymers and mixtures thereof.

8. The composition of claim 8, wherein the at least one anionic thickening polymer is chosen from ammonium acryloyldimethyltaurate/VP Copolymer, xanthan gum, and mixture thereof.

9. The composition of claim 1, wherein the at least one anionic thickening polymer is present from about 0.3 to about 2% wt. based on the total weight of the composition.

10. The composition of claim 1, wherein the at least one electrolyte is chosen from the group of α- and β-hydroxy acids, carboxylic acids, taurine, phosphoric acids, pyrophosphoric acid, cosmetically active organic acids, and mixture thereof.

11. The composition of claim 1, wherein the at least one electrolyte is chosen from glycolic acid, lactic acid, malic acid, citric acid, tartaric acid, mandelic acid, gluconic acid, ascorbic acid, and mixtures thereof.

12. The composition of claim 1, wherein the at least one electrolyte is present from about 3% wt. to about 30% wt. based on the total weight of the composition.

13. The composition of claim 1, wherein the at least one electrolyte is present from about 5% wt. to about 25% wt. based on the total weight of the composition.

14. The composition of claim 1 having a pH of about 5 to about 6.

15. A cosmetic composition comprising:
a) arginine;
b) at least one clay compound;
d) at least one anionic thickening polymer; and
wherein the composition is thermally stable and contains a high level of electrolytes.

16. A method for treating skin comprising applying a cosmetic composition of claim 1 to the skin.
